(19) United States
(12) Patent Application Publication
(10) Pub. No.: US 2019/0297968 A1
(43) Pub. Date: Oct. 3, 2019

(54) Glove With Visual Indicator

(71) Applicants: Ashwin George, Eagan, MN (US); Svetlana Zaydman, Eagan, MN (US)

(72) Inventors: Ashwin George, Eagan, MN (US); Svetlana Zaydman, Eagan, MN (US)

(21) Appl. No.: 16/368,019

(22) Filed: Mar. 28, 2019

Related U.S. Application Data
(60) Provisional application No. 62/649,860, filed on Mar. 29, 2018.

(51) Int. Cl. A41D 19/00; A41D 19/015; A61B 42/10

(52) U.S. Cl. CPC A41D 19/0062; A61B 42/10; A41D 19/01594

(57) ABSTRACT

A glove having an inside surface and an outside surface, for enclosing a hand, and having a wrist end and a visual marker on the inside surface at the wrist end of the glove to remind a wearer of the glove, as the glove is being removed and the visual marker is seen, to utilize proper glove removal procedure to prevent the soiled glove from contaminating the hands or wrist of the wearer.

### CROSS-REFERENCE TO RELATED APPLICATIONS

Not Applicable

### STATEMENT REGARDING FEDERALLY SPONSORED RESEARCH

Not Applicable

### FIELD OF THE INVENTION

The invention relates to a visual indicator placed on a peel-back point, such as the inside surface of the glove near the wrist end, which when seen reminds the wearer to utilize proper procedure to remove the glove to prevent contamination of the hands and/or wrist from medical material on the outside of the gloves.

### BACKGROUND OF THE INVENTION

Medical gloves are to be removed using proper procedure, which is often forgotten when removing the gloves. Examples of proper procedure for removing gloves can be found at https://www.cdc.gov/vhfiebola/pdftposter-how-to-remove-gloves.pdf, https.//www youtube.com/watch?v=S4gyNAsPCbU, http://www.safecare-gloves.com: http.//www.probloodborne.com/en/training video/glove-removal-and-disposal, and http://www.instructables.com/d/How-to-Remove-Gloves-Personal-Protective-Equipmen/, the entire contents of which are hereby incorporated by reference in their entirety.

The problems caused by improper glove removal is described here, http://www.cbc.ca/news/health/gown-gloves-germs-1.3267493, the entire contents of which are hereby incorporated by reference in their entirety.

What is needed is something to remind the wearer of the glove to use proper procedure for removal.

### BRIEF SUMMARY OF THE INVENTION

The invention is a glove having an inside surface, with a visual marker located on the inside surface near the wrist end of the glove. If the wearer forgets to use proper procedure to remove the glove, when they see the visual marker on the inside surface, near the wrist end of the glove, they will be reminded to use proper procedure and avoid contamination.

The visual marker can be a symbol or graphic, a color change, a colored line, a marker strip, or florescent material, which can be located in several spots around the inside periphery of the wrist end of the glove or extend entirely around the inside periphery of the glove.

### BRIEF DESCRIPTION OF THE DRAWINGS

FIG. 1 is a view of a glove showing a visual marker on the inside surface of the glove.

FIG. 2 is a view of the wrist end of a glove showing a visual marker extending around the entire inside periphery of the glove.

FIG. 3 is a view of the wrist end of a glove showing a visual marker which can be a different color than the glove material.

FIG. 4 is a view of the wrist end of a glove showing a visual marker which is a colored line extending around the entire inside periphery of the glove.

FIG. 5 is a view of the wrist end of a glove showing a visual marker which is a strip extending around the entire inside periphery of the glove.

FIG. 6 is a view of the wrist end of a glove showing a visual marker which is a section of fluorescent material.

FIG. 7 is a view of the wrist end of a glove showing a visual marker which is a graphic.

FIG. 8 is a view of the wrist end of a glove showing a visual marker which can extend from the wrist end of the glove, all the way to the finger and thumb holes on the inside of the glove, or any smaller portion thereof.

### DETAILED DESCRIPTION OF THE INVENTION

While this invention may be embodied in many forms, there are described in detail herein specific embodiments of the invention. This description is an exemplification of the principles of the invention and is not intended to limit the invention to the particular embodiments illustrated.

For the purposes of this disclosure, like reference numerals in the figures shall refer to like features unless otherwise indicated.

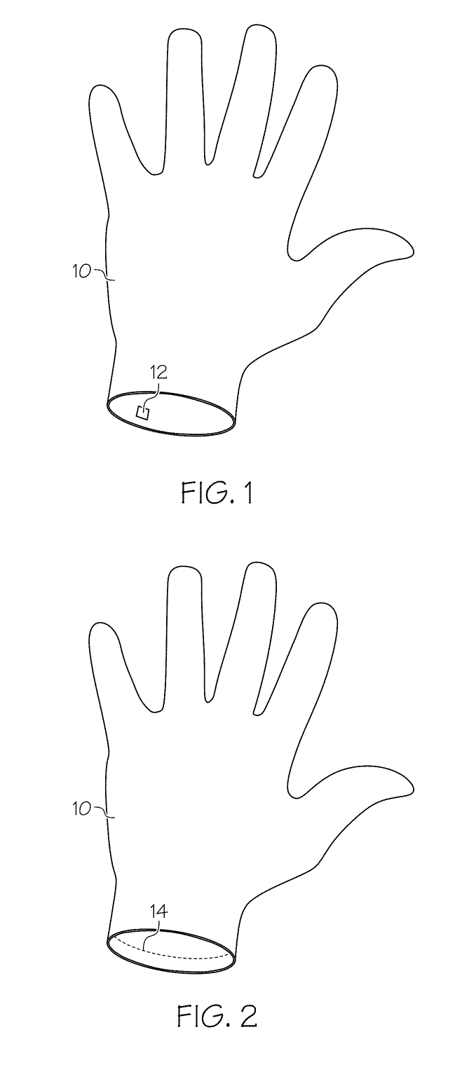

Referring now to FIG. 1, a glove is shown at 10, with a visual marker 12 located on the inside surface near the wrist end of the glove. The visual marker 12 can be a symbol or graphic, a color change in the glove material, a colored line extending around the inside periphery of the wrist end of the glove, a marker strip or florescent material. The visual marker 12 can be located preferably in the first ⅓ portion of the glove, between the wrist end and the finger portions, but could be located anywhere between the wrist end and the finger openings, and take all or any desired portion of the inside glove area between the wrist end and the finger and thumb holes.

FIG. 2 shows is a view of the wrist end of a glove showing a visual marker 14 extending around the entire inside periphery of the glove.

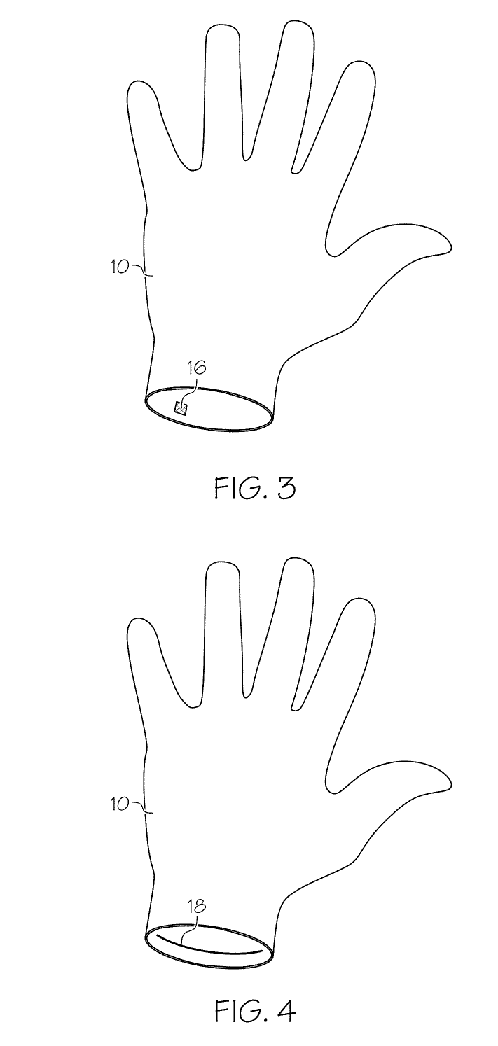

FIG. 3 shows a view of the wrist end of a glove showing a visual marker 16 which can be a different color than the glove material.

FIG. 4 is a view of the wrist end of a glove showing a visual marker 18 which is a colored line extending around the entire inside periphery of the glove.

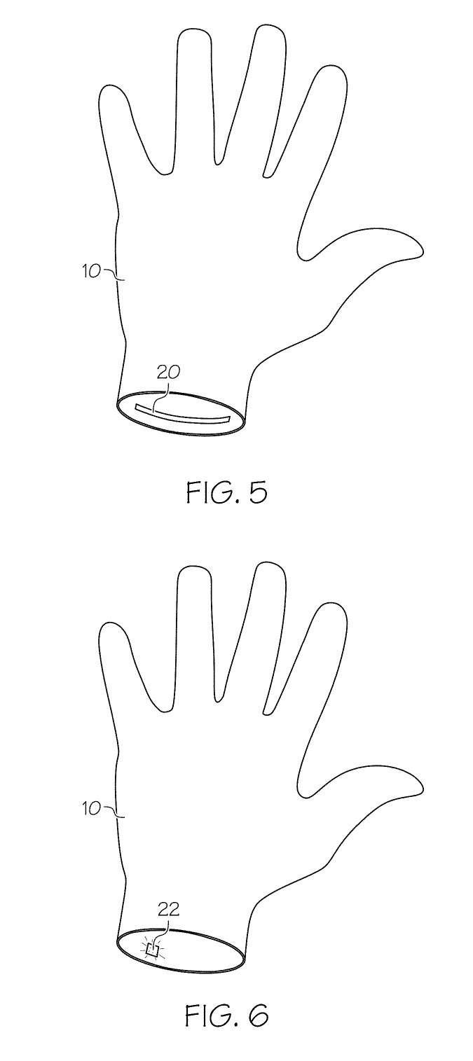

FIG. 5 is a view of the wrist end of a glove showing a visual marker 20 which is a strip extending around the entire inside periphery of the glove.

FIG. 6 is a view of the wrist end of a glove showing a visual marker 22 which is a section of fluorescent material.

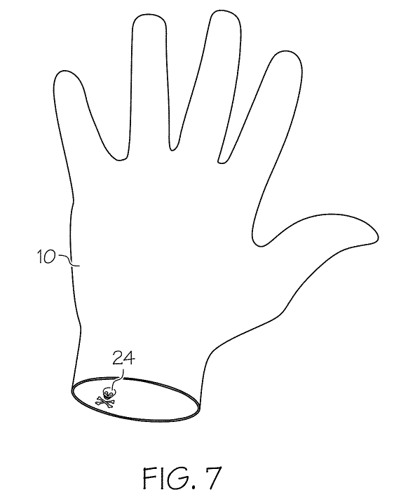

FIG. 7 is a view of the wrist end of a glove showing a visual marker 24 which is a graphic.

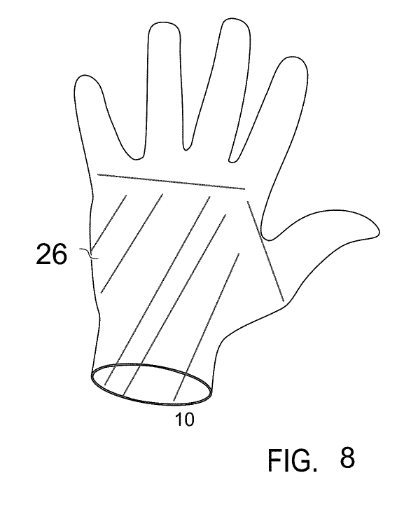

FIG. 8 is a view of the wrist end of a glove showing a visual marker 26 which can extend from the wrist end of the glove, all the way to the finger and thumb holes on the inside of the glove, or any smaller portion thereof.

As the wearer is beginning to remove the glove, they will see the visual indicator on the inside surface of the glove and be reminded to use proper procedure to remove the glove, thereby cutting down on contamination of the hand and/or wrist.

What is claimed is:

1. A glove comprising:
material having an inside surface and an outside surface, for enclosing a hand, and having a wrist end;
a visual marker on the inside surface at the wrist end of the glove to remind a wearer of the glove, as the glove is being removed and the visual marker is seen, to utilize proper glove removal procedure to prevent the soiled glove from contaminating the hands or wrist of the wearer.

2. The glove of claim 1 wherein the glove is a medical glove.

3. The glove of claim 1 wherein the visual marker extends around the periphery of the wrist end of the inside surface of the glove.

4. The glove of claim 3 wherein the visual marker is a section of glove material having a different color than the surrounding glove material color.

5. The glove of claim 1 wherein the visual marker is a colored line.

6. The glove of claim 1 wherein the visual marker is a marker strip.

7. The glove of claim 1 wherein the visual marker is a fluorescent material.

8. The glove of claim 1 wherein the visual marker is a symbol or graphic.

9. A glove comprising:
material having an inside surface and an outside surface, for enclosing a hand, and having a wrist end;
a visual marker on the inside surface of the glove to remind a wearer of the glove, as the glove is being removed and the visual marker is seen, to utilize proper glove removal procedure to prevent the soiled glove from contaminating the hands or wrist of the wearer.

10. The glove of claim 9 wherein the visual marker extends from the wrist end of the glove to the finger holes and thumb hole, or any smaller portion thereof.
